(19) United States
(12) Patent Application Publication
(10) Pub. No.: US 2026/0348800 A1
(43) Pub. Date: Nov. 5, 2026

(54) METHOD FOR PRODUCING REFERENCE MATERIALS

(71) Applicant: Independent Mineral Standards Pty Ltd., Bayswater, Western Australia (AU)

(72) Inventors: Bruce Edward John ARMSTRONG, Wembley, Western Australia (AU); John CARTER, Marmion, Western Australia (AU)

(21) Appl. No.: 18/997,548

(22) PCT Filed: Jun. 30, 2023
(86) PCT No.: PCT/AU2023/050610

§ 371 Date: Jan. 22, 2025

(30) Foreign Application Priority Data
Jul. 29, 2022 (AU) 2022902136

(51) Int. Cl. G01N 1/28; C04B 14/30; C04B 22/06; C04B 22/08; C04B 28/02; C04B 41/00; C04B 103/10; G01N 33/24; G01N 33/38

(52) U.S. Cl. CPC G01N 1/286; C04B 14/308; C04B 22/062; C04B 22/08; C04B 28/021; C04B 41/0072; C04B 41/009; G01N 33/24; G01N 33/383; C04B 2103/10; G01N 2001/2866; G01N 2001/2893

(57) ABSTRACT

The present invention relates to a method for the production of a reference material, the method comprising the steps of: combining at least one target substance, one or more binding reagents and an activator solution to form a precursor slurry; curing the precursor slurry to form a solid material; and crushing the solid material to produce a reference material.

### TECHNICAL FIELD

In accordance with the present invention, there is provided a method for the production of a reference material. More specifically, forms of the present invention provide for a method for the production of a reference material which has a customisable particle size. The present invention further provides for a reference material produced by this method.

### BACKGROUND ART

The following discussion of the background art is intended to facilitate an understanding of the present invention only. The discussion is not an acknowledgement or admission that any of the material referred to is or was part of the common general knowledge as at the priority date of the application.

Sample analysis is used across many industries to provide information on the chemical makeup of materials and concentrations of target measurands. The sampling methodologies and analytical techniques used are strictly regulated to ensure accurate results are produced. Quality control checks are also conducted during the analysis of samples to ensure that the reported result is reflective of the actual concentration of the target measurand. This prevents inaccurate results that may be caused by uncalibrated, miscalibrated, malfunctioning, broken, or worn-out instruments and to ensure that the correct sampling procedures have been adhered to. Quality control checks commonly include the periodic testing of a reference material which has known physical, chemical or mineralogical features. The comparison of the measured values against the actual values from the reference material is used to check that the results are within acceptable levels of variance. Analysis of a reference material for batch or continuous laboratory operations may be used to determine bias in the analysis and/or trends over time. Reference materials may also be used in the analysis of other physical properties, such as composition and mineralogy.

The majority of the analytical techniques to determine the concentration of a target measurand require the sample have a small particle size. This is largely because the analytical technique usually digests the sample and this isn't possible to perform on large particles, which do not digest or will digest slowly. Traditional reference materials are provided at a small particle size for use in such analytical techniques without the need for further processing. The small particle size of traditional reference samples limits their use in analytical techniques that accept a larger particle size.

Traditional reference materials are produced using a naturally occurring ore containing the target metal of interest. However, the target metal is not commonly homogenously dispersed through the ore. Crushing the natural ore to a smaller particle size improves the homogeneity of the target metal through the reference material. If the natural ore is not crushed to a small particle size to improve homogeneity, the reference material is likely to produce analysis results that vary widely. This is especially true for natural ores where the target metals are localised to within only a few grains of the ore. Examples of such ores include gold and precious metals ores. Such ores are often referred to as ‘nuggety’ ores, where the target metal is localised into nuggets. Crushing of such ores to a particle size especially of −5 mm or −3 mm and in most cases pulverizing to a particle size as low as 100 μm can still result in the amount of the target metal in each analytical aliquot to vary widely. Such ores are not suitable for use as reference materials unless the particle size has been reduced significantly to improve homogeneity.

Typical samples presented to the laboratory from the field are rock core, chips, drill samples, or various field rock and soil samples that are from 0-10 kg in mass and have particle sizes up to 40 mm. The received samples require sub-sampling steps and preparation steps to produce a fine powder suitable for analysis by the laboratory. Variances in either the sub-sampling steps or the preparation steps can lead to errors in the final concentration. However, traditional reference samples may only be inserted into the analytical procedure to provide a quality check once the sample has a similar particle size to the reference sample. This will provide no indication of any errors that were introduced during the previous sub-sampling steps or sample preparation steps.

Throughout this specification, unless the context requires otherwise, the word “comprise” or variations such as “comprises” or “comprising”, will be understood to imply the inclusion of a stated integer or group of integers but not the exclusion of any other integer or group of integers.

### SUMMARY OF INVENTION

In accordance with the present invention there is provided a method for the production of a reference material, the method comprising the steps of:
combining at least one target substance, one or more binding reagents and an activator solution to form a precursor slurry;
curing the precursor slurry to form a solid material; and
crushing the solid material to produce a reference material.

The inventors have found that the method of the present invention allows for a target substance to be substantially homogenously distributed throughout a solid material. This solid material may then be crushed using typical sample preparation techniques to provide a reference sample material. By carefully controlling the amount of each substance in the precursor slurry, the concentration of the target substance in the final reference material is accurately known.

In one form of the present invention, the target substance contains a target metal.

In one form of the present invention, the one of more target substances are provided as a solid material. In one embodiment, the solid material may be soluble in a constituent of the slurry. In an alternative embodiment, the solid material is insoluble.

In an alternative form of the present invention, the one or more target substances are provided in a carrier solution. In one embodiment, the target substances are solubilised in the carrier solution. In an alternative form of the present invention, the target substances are suspended in a carrier solution. In an alternative form of the present invention, the target substance is provided as a complex ion which is soluble in a carrier solution. Preferably, the complex ion is formed from the target substance and a suitable complexing agent. In one form of the present invention, the carrier solution is the activator solution.

In an alternative form of the present invention, the target substance is a constituent of at least one of the binding reagents. In one form of the present invention, the binding reagent comprising the target substance is a pulverised natural ore comprising the target substance. Alternatively, the binding reagent comprising the target substance is a pulverised synthetic material comprising the target substance.

Throughout this specification, unless the context requires otherwise, the term “pulverised”, will be understood to refer to a material with a particle size below 100 μm. Such materials typically result from the fine grinding of a material to the required size followed by screening to remove oversize particles.

Throughout this specification, unless the context requires otherwise, the term “crushed”, will be understood to refer to a material with a particle size above 100 μm. Such materials typically result from the crushing of a material to the required size followed by screening to remove oversize particles.

In one form of the present invention, the binding reagents are selected from one or more of geopolymer composite binders, hydraulic binders and refractory cement binders. In one form of the present invention, the binding reagents are selected from synthetic materials, naturally occurring materials or combinations of both.

In one form of the present invention, the one or more binding reagents are provided as a dry composition. Preferably, the one or more binding reagents are particulate.

In one form of the present invention, the activator solution activates the one or more binding reagents. Throughout this specification, unless the context requires otherwise, the word “activate” or variations such as “activates” or “activating”, will be understood to refer to the initiation of the binding of the one or more binding reagents to form a solid material. In one form of the present invention, the activator solution is an aqueous solution. In an alternative form of the present invention, the activator solution comprises an activator substance.

In one form of the present invention, one or more aggregates are included in the precursor slurry. Preferably, the aggregates are selected from rock, sand, silica, siliceous minerals, silicon dioxide, lime, limestone, chalk, talc, blast furnace slag, fly ash, refractory materials and clay. Preferably, the one or more aggregates are crushed.

In one form of the present invention, the target substance is a constituent of at least one of the aggregates. In this embodiment, the aggregate comprising the target substance is a pulverised material. Preferably, the aggregate comprising the target substance is a pulverised natural ore comprising the target substance. Alternatively, the binding reagent comprising the target substance is a pulverised synthetic material comprising the target substance.

In one form of the present invention, one or more additives are included in the precursor slurry. In one form of the present invention, the additives are selected from retardants, viscosity modifiers, rheological modifiers, anti-foaming agents, surfactants and plasticizers.

In one form of the present invention, the one or more binding reagents, the aggregates and the additives are combined prior to the contact of the activator solution.

In one form of the present invention, the at least one target substance is combined with the one or more binding reagents prior to the contact of the activator solution. In an alternative form of the present invention, the at least one target substance is combined with the one or more binding reagents simultaneously to the contact of the activator solution. In an alternative form of the present invention, the at least one target substance is combined with the one or more binding reagents following the contact of the activator solution.

In one form of the present invention, the precursor slurry is subjected to a mixing step.

In one form of the present invention, the precursor slurry is poured into a mould.

In one form of the present invention, curing the precursor slurry comprises a drying step. In one form of the present invention, the drying step is conducted at elevated temperatures. Additionally or alternatively, curing the precursor slurry comprises an activation step.

Preferably, the crushing step further comprises a sizing step.

In one form of the present invention, the crushed material may be combined with a filler material.

In accordance with a further embodiment of the present invention, there is provided a reference material produced using the method discussed above.

### BRIEF DESCRIPTION OF THE DRAWINGS

Further features of the present invention are more fully described in the following description of two non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 shows a box a whisper graph of the results of the photon analysis of a first sample prepared according to Example 1;

FIG. 2 shows a box a whisper graph of the results of the photon analysis of a second sample prepared according to Example 1;

FIG. 3 shows a box a whisper graph of the results of the photon analysis of a third sample prepared according to Example 1;

FIG. 4 shows a plot of sample grade vs the recorded homogeneity relative standard deviation of the samples prepared according to Example 1;

FIG. 5 shows the results of the sub-sampling analysis conducted to represent a good sub-sampling routine;

FIG. 6 shows the results of the sub-sampling analysis conducted to represent a poor sub-sampling routine;

FIG. 7 shows a plot comparing different dilution ratio of geopolymer to basalt filler measured as between unit variance created by different sub-sampling routines;

FIG. 8(a)-(c) shows a plot comparing the results of characterisation studies carried out by different laboratories; and

FIG. 9 shows a plot comparing the analytical results following the reanalysis of samples prepared by different laboratories.

### DESCRIPTION OF EMBODIMENTS

The present invention provides a method for the production of a reference material. The final reference material comprises a target substance that is substantially homogenously dispersed through a solid material at a known concentration. The method of the present invention allows for the production of a bulk solid material that may undergo further physical processing to produce a reference material with a desired particle size. Advantageously, this allows the particle size of the reference material to be tailored to suit the particular analytical technique used during sampling.

The method generally comprises the combination of at least one target substance with one or more binding reagents and an appropriate activator solution to form a precursor slurry. The one or more binding reagents are in a form which allows the target substance to be homogenously dispersed therethrough and will only begin to bind together upon contact with a suitable activator solution. The precursor slurry is then cured to form a solid material. Combining the target substance with the one or more binding reagents prior to the curing of the solid material allows the target substance to be distributed throughout the formed solid material. The solid material may then be crushed to an appropriate particle size for use as a reference material.

The one or more binding reagents are selected from materials that comprise discrete particles that will bind together upon activation. This allows the target substances to be dispersed throughout the binding reagents prior to or during the binding process.

In a preferred embodiment of the present invention, the binding reagents are solid particles. Still preferably, the one or more binders may be provided as a dry composition.

In one form of the present invention, the one or more binder reagents comprise geopolymer composite binders. Geopolymer composite binders comprise pozzolan materials that will bind upon reaction with an alkaline activator solution. Pozzolan materials are siliceous or siliceous and aluminous. Suitable pozzolan materials include fly ash, metakaolin, clays, calcined clays, fumed silica, volcanic ashes and zeolites.

Preferably, where the one or more binder reagents comprise geopolymer composite binders, the activator solution is an alkaline solution. More preferably, the alkaline solution is selected from sodium hydroxide, sodium silicate, potassium hydroxide and potassium silicate.

In one form of the present invention, the one or more binder reagents comprise hydraulic binders. As would be appreciated by a person skilled in the art, hydraulic binders are typically finely ground inorganic material that forms a paste when mixed with water and hardens by means of hydration. Preferably, the hydraulic binders are selected form one or more of cement, portland cements, chalk, talc, blast furnace slag, fly ash and clays.

Preferably, where the one or more binder reagents comprise hydraulic binders, the activator solution is an aqueous solution.

In one form of the present invention, the one or more binder reagents comprise refractory cement binders. As would be understood by a person skilled in the art, refractory cement binders are a specialised class of hydraulic binders which bind to form a solid material which is resistant to decomposition by heat. Preferably, the refractory cement binders comprise calcium and alumina. More preferably, the refractory cement binders are selected from one or more of aluminates, phosphates, silicates and sulphates. Still preferably, the refractory cement binders comprise one or more hydraulic calcium aluminates. Suitable calcium aluminate cements include monocalcium aluminate, dodecacalcium hepta-aluminate, monocalcium dealuminate. It is envisaged that the refractory cement binders may include other phases together with the hydraulic calcium aluminates, for example, silicon oxide, aluminium oxide, iron oxide, calcium oxide, magnesium oxide, sodium oxide, potassium oxide and titanium oxide.

Preferably, where the one or more binder reagents comprise refractory cement binders, the activator solution is an aqueous solution.

In one form of the present invention, one or more aggregates are included in the precursor slurry. Preferably, the aggregates are inert solid particles that participate in the binding of the one or binding reagents. Once the binding reagents are suitably activated, they will bind the aggregate particles together into the solid material. activated.

As would be appreciated by a person skilled in the art aggregates are used to provide particular physical properties to the final solid product. The choice and amount of aggregates used is dependent on the binding reagents used.

Preferably, where the one or more binder reagents comprise geopolymer composite binders, the aggregates are selected from sand, silica, silicon dioxide, feldspar, granite, basalt or any highly siliceous mineral.

Preferably, where the one or more binder reagents comprise hydraulic binders, the aggregates are selected from hydrated lime, limestone, chalk, talc, blast furnace slag, fly ash, sand and clay.

Preferably, where the one or more binder reagents comprise refractory cement binders, the aggregates are selected from alumina, alumina-zirconia, aluminum silicate or sillimanite, calcium aluminate, calcium silicate, carbon or graphite, chromia or chromite, dolomite, kaolin or fire clay, magnesia or magnesite, magnesium silicate or forsterite, mullite, porcelain, silicon carbide, silica or fused silica, spinel, titania or titanate, yttria, zircon, and zirconia.

In one embodiment of the present invention, the total mass of the one or more target substances in the precursor slurry is 0.0001-30% by weight. The amount of each target substance in the precursor slurry can be tailored to provide the desired final concentration in the produced reference material. This will depend on the target substance itself and the expected/typical concentration of the target substance in the samples being analysed. For example, base metal ores are typically of a higher grade than gold ores. In one embodiment, the total mass of the one or more target substances in the precursor slurry is 0.0001-20% by weight. In one embodiment, the total mass of the one or more target substances in the precursor slurry is 0.0001-15% by weight. In one embodiment, the total mass of the one or more target substances in the precursor slurry is 0.0001-5% by weight. In one embodiment, the total mass of the one or more target substances in the precursor slurry is 0.0001-1% by weight. In one embodiment, the total mass of the one or more target substances in the precursor slurry is 0.0001-0.5% by weight.

In an alternative form of the present invention, the binding reagents are provided in a carrier solution. Suitable binding reagents include monomer and polymer materials that will polymerise or crosslink upon activation.

In one form of the present invention, one or more additives are included in the precursor slurry. The one or more additives are used to alter the physical properties of the final solid and/or the curing process. Suitable additives include retardants, viscosity modifiers, rheological modifiers, anti-foaming agents, surfactants and plasticisers. Additives may also be used to alter the physical appearance of the solid material, such as colour, porosity and texture.

The target substance preferably contains a target metal of interest in the sampling procedure. For example, the target substance may contain Au, Ag, Pt, Pd, Cu, Ni, Co, Pb, Zn, Li, or others. It is envisaged that the target substance may be provided in any form which allows the target substance or target metal to be substantially homogeneously dispersed through the precursor slurry.

In one embodiment, the target substance is a solid particulate material. The target substance may be provided in its elemental form, an ionic compound or a mineral. Where the target substance is insoluble to the constituents of the precursor slurry, the target substance must have a sufficiently sized particle size to allow it to be distributed substantially homogeneously throughout the precursor slurry. In one embodiment, the particle size is less than 100 μm. In one embodiment, the particle size is less than 80 μm. In one embodiment, the particle size is less than 50 μm. In one embodiment, the particle size is less than 50 μm. In one embodiment, the particle size is between 1 and 100 μm. In a preferred embodiment, the target substance is nanoparticulate.

In an alternative embodiment, the target substance is provided as a constituent of at least one of the binding reagents. It is envisaged that in certain binding reagents may contain target metals and that such target metals may be utilised as the target substance in the present invention. In such embodiments, the binding reagent requires sufficient grinding to ensure a homogenous distribution of the target metal throughout the precursor slurry and the resulting solid material. In one embodiment, the particle size of the binding reagent is less than 100 μm. In one embodiment, the particle size of the binding reagent is less than 80 μm. In one embodiment, the particle size of the binding reagent is less than 50 μm. In one embodiment, the particle size of the binding reagent is less than 50 μm. In one embodiment, the particle size of the binding reagent is between 1 and 100 μm. In a preferred embodiment, the binding reagent is nanoparticulate. The concentration of the target metal in the binding reagent is analysed prior to mixing with other materials such that the final concentration of the target metal is the precursor slurry is known.

Many natural ores contain minerals that exhibit hydraulic properties. The fine grinding of such ores would allow the use of the pulverised material as at least one of the binding reagents of the present invention. In some instances, the pulverised ore alone may be suitable for contact with the activator solution to produce the solid material. Alternatively, additional binding reagents may be added to the pulverised material prior to contact with the activator solution to produce the solid material. It is envisaged that the incorporation of a natural ore into the reference material of the present invention would allow the characteristics of the reference material to be better matched with the characteristics of the sample being analysed, thereby providing additional confidence in the test results.

Many synthetic materials exhibit hydraulic properties. Examples of such materials include fly ash, combustion residuals, product residues, kiln dusts, furnace dusts, slags, ashes and other industrial wastes. These materials often include metals that may be used as the target substance of the present invention. The fine grinding of such materials would allow the use of the pulverised material as at least one of the binding reagents of the present invention. In some instances, the pulverised material alone may be suitable for contact with the activator solution to produce the solid material. Alternatively, additional binding reagents may be added to the pulverised material prior to contact with the activator solution to produce the solid material. It is envisaged that the incorporation of such synthetic materials into the reference material of the present invention may allow the characteristics of the reference material to be better matched with the characteristics of the sample being analysed, thereby providing additional confidence in the test results.

In an alternative embodiment, the target substance is provided as a constituent of at least one of the aggregates. It is envisaged that certain aggregates may contain target metals and that such target metals may be utilised as the target substance in the present invention. The concentration of the target metal in the aggregate is analysed prior to mixing with other materials such that the final concentration of the target metal is the precursor slurry can be calculated. Many natural mineral materials contain target metals. Similarly, many synthetic materials contain target metals. It is envisaged that the fine grinding of such materials would allow the use of the pulverised material as at least one of the aggregates of the present invention. In this embodiment, the pulverised material is mixed with the one or more binding reagents and the activator solution to produce the solid material. In such embodiments, the aggregate requires sufficient grinding to ensure a homogenous distribution of the target metal throughout the precursor slurry. In one embodiment, the particle size of the aggregate is less than 100 μm. In one embodiment, the particle size of the aggregate is less than 80 μm. In one embodiment, the particle size of the aggregate is less than 50 μm. In one embodiment, the particle size of the aggregate is between 1 and 100 μm. In a preferred embodiment, the aggregate is nanoparticulate. It is envisaged that the preferred aggregate material selected to be included in the reference material would be a main constituent of the material being sampled. As discussed above, it is envisaged that the incorporation of an aggregate containing the target metal into the reference material of the present invention would allow the characteristics of the reference material to be better matched with the characteristics of the sample being analysed, thereby providing additional confidence in the test results.

In an alternative embodiment, the target substance may be provided as a solid soluble salt. It is envisaged that the target substance will solubilise in the precursor slurry, allowing the target substance to be dispersed throughout the precursor slurry. The soluble salt may be soluble in one the other constituents of the precursor slurry, for example water or the alkaline activator solution. Alternatively, a suitable solvent may also be included in the precursor slurry to solubilise the target substance. Where the target substance is a solid soluble salt, the particle size should be selected to ensure that the target substance fully solubilises in the precursor slurry prior to curing.

In an alternative embodiment, the target substance is distributed in a carrier solution. Preferably, the target substance is dissolved in a suitable solvent solution. Alternatively, the target substance may be a suspension or colloid suspension. Alternatively, a soluble complex ion may be formed between the target substance and a suitable complexing agent, for example cyanide.

In a preferred form of the present invention, the target substance is provided as an ion in solution. This has been found to improve the homogeneity of the final solid product.

In one embodiment of the present invention, the precursor slurry comprises:
50-80% by weight binding reagents;
20-50% by weight activator solution; and
0.0001-30% by weight target substances.

In an alternative embodiment of the present invention, the precursor slurry comprises:
30-80% by weight binding reagents;
10-70% by weight aggregates;
20-50% by weight of an activator solution; and
0.0001-30% by weight target substances.

In a preferred form of the present invention, where the one or more binder reagents comprise geopolymer composite binders, the precursor slurry comprises:
30-80% by weight binding reagents;
10-70% by weight aggregates;
20-50% by weight of an alkaline activator solution; and
0.0001-30% by weight target substances.

More preferably, where the geopolymer composite binders comprise fly ash, the precursor slurry comprises:
70-75% by weight binding reagents;
2-10% by weight aggregates;
10-30% by weight of an alkaline activator solution; and
0.0001-18% by weight target substances.

More preferably, where the geopolymer composite binders comprise metakaolin, the precursor slurry comprises:
15-25% by weight binding reagents;
40-50% by weight aggregates;
30-40% by weight of an alkaline activator solution; and
0.0001-15% by weight target substances.

In a preferred form of the present invention, where the one or more binder reagents comprise hydraulic binders, the precursor slurry comprises:
20-60% by weight binding reagents;
30-70% by weight aggregates;
5-30% by weight of an aqueous activator solution; and
0.0001-30% by weight target substances.

More preferably, where the one or more binder reagents comprise hydraulic binders, the precursor slurry comprises:
15-25% by weight binding reagents;
55-65% by weight aggregates;
5-15% by weight of an aqueous activator solution; and
0.0001-25% by weight target substances.

In a preferred form of the present invention, where the one or more binder reagents comprise a pulverised natural ore comprising a target substance, the precursor slurry comprises:
50-95% by weight pulverised natural ore; and
5-50% by weight aqueous activator solution.

In a preferred form of the present invention, where the one or more binder reagents comprise a pulverised synthetic material comprising a target substance, the precursor slurry comprises:
50-95% by weight pulverised synthetic material; and
5-50% by weight aqueous activator solution.

In a preferred form of the present invention, where the one or more binder reagents comprise refractory cement binders, the precursor slurry comprises:
10-70% by weight binding reagents;
0-70% by weight aggregates;
10-50% by weight of an aqueous activator solution; and
0.0001-30% by weight target substances.

The contact of the activator solution and the one or more binding reagents will initiate the binding of the binding reagents. It is important to ensure that all the components of the precursor slurry are added before the precursor slurry begins to solidify. Preferably, one or more of the components may be mixed together prior to the addition of the activator solution. For example, one or more binding reagents, the aggregates and the additives may be combined prior to the addition of the activator solution. It is envisaged that the target substance may be combined prior to the addition of the activator solution or following the addition of the activator solution. It is further envisaged that the activator solution may be the carrier solution for the target substance. For example, where the activator solution is in the form of an aqueous solution, the target substance may be dissolved in the aqueous solution. Those skilled in the art would understand that the order of addition may depend on the particular binders and other substituents used and can tailor this to ensure that a homogenous mixture is provided. In embodiments where the one or more binders comprise geopolymer composite binders, hydraulic binders or refractory cement binders, it is envisaged that the binders, aggregates, additives and activator solution may be first mixed to provide a homogenous slurry. The target substance may then be added whilst the slurry is agitated.

The combination of the precursor slurry substituents may be performed in any suitable mixing or agitation device known in the art. Suitable mixing devices include drum mixers, cement mixers and paddle mixers. Those skilled in the art would understand that the choice of mixing apparatus may depend on the binders used.

Once the substituents of the precursor slurry have been sufficiently homogenised, the precursor slurry is preferably transferred to a mould. The precursor slurry may be transferred to a single mould or multiple moulds. The mould is constructed from a material inert to the precursor slurry. Any suitable shape mould may be utilised.

The precursor slurry is then cured to form a solid material. The curing process is dependent on the binders used in the precursor slurry.

In one embodiment, the curing process comprises a standing step. As would be appreciated by a person skilled in the art, the contact of the activation solution with certain binders will initiate a chemical reaction that will bind the binding reagents. A standing step will allow the binding reaching to complete. The length of time required is dependent on the binders and any additives that may be included.

In one embodiment, the curing process comprises a drying step. The drying step may be conducted at ambient temperatures or elevated temperatures. In one embodiment, the drying step may comprise multiple drying stages operating at increasing temperatures. In one embodiment, the drying step is conducted at temperatures between 20 °C-250 °C.

In one embodiment, the curing process comprises an activation step. As would be appreciated by a person skilled in the art, binding reagents may require chemical or physical activation in order to cure the binder. Suitable activation steps include high temperature treatments, steam treatments and irradiation with electromagnetic waves. The activation step may be conducted prior to a drying step or following a drying step.

In a preferred form of the present invention, where the one or more binder reagents comprise geopolymer composite binders, the curing step comprises drying of the precursor slurry at multiple stages between ambient temperature and 250 °C for a period of 24-96 hrs. Preferably, the drying step is conducted in a temperature controlled oven or kiln. Those skilled in the art would recognise that increasing the temperature of the curing step can lower the required curing time. For example, the curing step could be conducted at 50 °C for 72 hrs, at 70 °C and 48 hrs, or at 100 °C for 24 hrs.

In a preferred form of the present invention, where the one or more binder reagents comprise hydraulic binders, the curing step comprises allowing the precursor slurry to stand at room temperature for between 1-3 days. Those skilled in the art would be able to recognise that different hydraulic binders have different curing rates.

In a preferred form of the present invention, where the one or more binder reagents comprise refractory cement binders, the curing step comprises allowing the precursor slurry to stand at room temperature, followed by a calcination step conducted at 1500 °C. Preferably, the precursor slurry is allowed to stand at room temperature for 1-3 days. Preferably, the calcination step is conducted for 1-5 hours.

Following the completion of the curing process, the solid material is removed from any mould used. The solid material is then directed to a crushing step to produce a particulate reference material. Any suitable crushing apparatus may be used to reduce the particle size of the solid material. Suitable apparatus include jaw crushers, cone crushers, impact crushers and gyratory crushers. Preferably, the crushing step further comprises a sizing step to ensure the reference material has a specified maximum particle size.

In one embodiment, the maximum particle size of the reference material is 10 mm. In one embodiment, the maximum particle size of the reference material is 9 mm. In one embodiment, the maximum particle size of the reference material is 8 mm. In one embodiment, the maximum particle size of the reference material is 7 mm. In one embodiment, the maximum particle size of the reference material is 6 mm. In one embodiment, the maximum particle size of the reference material is 5 mm. In one embodiment, the maximum particle size of the reference material is 4 mm. In one embodiment, the maximum particle size of the reference material is 3 mm. In one embodiment, the maximum particle size of the reference material is 2 mm. In one embodiment, the maximum particle size of the reference material is 1 mm.

In one embodiment, the minimum size of the reference material is not controlled. Preferably, the crushed material is passed through an appropriate sizer or screen to remove oversize particles. In this embodiment, fines are not removed.

In an alternative embodiment, the minimum size of the reference material is controlled. Preferably, the minimum size of the reference material is at least 100 um. Preferably, a sizer or screen is used to remove particle fines from the crushed material.

In one embodiment, the particle size of the reference material is between 100 um and 10 mm. In one embodiment, the particle size of the reference material is between 100 um and 8 mm. In one embodiment, the particle size of the reference material is between 100 um and 6 mm. In one embodiment, the particle size of the reference material is between 100 um and 4 mm. In one embodiment, the particle size of the reference material is between 100 um and 3 mm. In one embodiment, the particle size of the reference material is between 100 um and 2 mm.

The main advantage of the present invention is that there is no limitation on the particle size of the reference material and so it may be suited to meet the specifications of the sampling routine used. The inventors have found that this will allow the particle size of the reference material to be matched to the particle size of the raw sample being investigated. This will allow the reference material to be inserted into the sampling routine at a much earlier stage than traditional small particle size reference materials. The reference material can then be subjected to the same sub-sampling steps and sample preparation steps as the raw sample. Analysis of the reference material following any of the sub-sampling steps or sample preparation steps may be used to track any errors introduced in that step.

A further advantage of the present invention is that the particle size of the reference material may be tailored to suit the particular analysis method used. As would be appreciated by a person skilled in the art, many different analytical techniques may be used to determine the concentration of a target substance in a sample or any other physical property of a sample. The majority of these analytical techniques require a sample with a small particle size. Traditional reference materials are typically provided at a small particle size to allow them to be used in such techniques. However, other analytical techniques may allow a sample with a larger particle size to be used. For example, analytical techniques using high energy X-rays may only require a particle size of 2-3 mm. However, such high energy X-ray techniques also required a much larger sample volume, typically at least 500 g. Whilst traditional reference materials may still be used in such techniques, the increased volume required makes their use uneconomic. Furthermore, there is a risk that a bias may be introduced where there is a disparity between the particle size of the sample and the particle size of the reference material. Whilst this may be negated by crushing the sample to a smaller particle size, this increases the risk of other sampling errors may be introduced. The reference material of the present invention may be crushed to meet the specifications of large particle size analytical techniques or pulverised to a small particle size for other analytical techniques.

The physical properties of the reference material will depend on the binders used, together with any aggregates or additives. The inventors have found that the choice of these materials may be influenced by the analytical technique used in the sampling routine. It is important that the binders and other components of the reference material are amenable to the analytical technique and do not interfere with the analysis of the target substance. For example, kaolin binders have been found to be preferrable for use with wet chemical assay techniques and fly ash binders have been found to be preferrable for use with fire assay techniques.

In one embodiment, the produced reference material is directly used in a sampling routine. In an alternative embodiment, the produced reference material may be mixed with a filler material. It is envisaged that by mixing a known amount of the reference material with a known amount of filler material, the overall volume of the reference material may be increased without impacting the homogeneity of the reference material. The filler material may be selected from natural or synthetic materials and preferably has similar physical properties to the reference material. Suitable natural materials include crushed rocks, ores and minerals, such as basalt, feldspar, quartz or granite. Suitable synthetic materials include crushed cement, concrete or geopolymer.

In one embodiment, one or more of the physical properties of the filler may be selected to be different to the reference material. For example, the particle size distribution or the specific gravity of the filler material may be different to the reference material. The inventors have found that these differences may be used to highlight errors or poor practices during sub-sampling steps or sample preparation steps, which may cause a bias or failure of the reference material. This is particularly the case for sample splitting and dividing methods that can easily bias the sample if all particles in the reference material are not given an equal opportunity to be selected in the next sample stage.

In a preferred embodiment, the filler material does not contain any of the target substances. By providing a filler material without the target substance, the concentration of the target metal in the reference material may be simply diluted according to the ratio of reference material to filler material. For example, the reference material may contain 25 g/t of Au, but when it is mixed or combined with basalt containing 0 g/t of Au, the final product has a concentration of 2.5 g/t.

In one embodiment, the reference material is mixed with the filler material in a ratio of at least 1:20. In one embodiment, the reference material is mixed with the filler material in a ratio of at least 1:15. In one embodiment, the reference material is mixed with the filler material in a ratio of at least 1:10. In one embodiment, the reference material is mixed with the filler material in a ratio of at least 1:5.

In one embodiment, the reference material is mixed with the filler material in a ratio between 1:25 and 1:5. In one embodiment, the reference material is mixed with the filler material in a ratio between 1:20 and 1:10.

### Example 1

Three separate batches of a reference material having different gold concentrations were prepared in accordance with the present invention. Each batch comprised the components detailed in Table 1, where fly-ash was selected as the geopolymer binding reagent and NaOH is used as an activator.

TABLE 1
Components of each test batch

| Material | Weight (Kg) | Volume (L) |
|---|---|---|
| FlyAsh | 281.1 | 351.42 |
| Magnetite | 15.8 | 3.06 |
| 50% NaOH | 27.7 | 18.30 |
| Na-Silicate | 71.3 | 50.91 |
| Water | 19.6 | 19.62 |

To prepare an activator solution, the NaOH solution, Na-silicate and water were initially mixed together and allowed to stand for ~24 hrs. The fly-ash and magnetite were then added to a mixer and were mixed for 1-2 minutes to ensure a homogenous dry mixture. The activator solution was then added to the mixer to combine with the dry mixture and form a slurry. The mixer was stopped at regular increments to check for dry clumps of mixture along the edges, bottom, or grooves of the mixer. This was repeated until no dry clumps remained. The mixture was then vibrated for 1 minute and any resulting surface carbon is scraped off. This was repeated 6 times until minimal carbon is present. Gold was then added to each batch at a target concentration of 10-15 g/T.

The mixture was then combined for ~5 minutes in the mixer to ensure homogenous mixture. The precursor slurry was then poured into a tray mould and placed into the oven at 100 °C for 24 hrs. The resulting solid material was then crushed and screened to −3 mm.

The resulting solid material was then mixed with a ballast filler material of basalt, which had also been crushed and screened to −3 mm, in ratios of approximately 1:20, 1:10 and 1:4. The resulting batches had gold concentrations shown to Table 2.

TABLE 2
Gold added to each test batch

| Batch Reference | IMS219 | IMS220 | IMS221 |
|---|---|---|---|
| Target Au (g/T) | 0.33 | 0.98 | 2.65 |

The resulting material was then tested for homogeneity. Reference material between-unit and within-unit material homogeneity is measured against the analytical technique uncertainty, and must be less than that of the intended analytical technique to be considered homogeneous. Separation of uncertainty source between material and analytical technique is derived by test design, typically using repeated analysis and analysed via Analysis of variance (ANOVA).

Between unit uncertainty is derived by Formula (1):

$${s}_{bb}^{2} = max ( \frac{{M}_{between} - {M}_{within}}{{n}_{0}} , 0 ) \qquad ( 1 )$$

where nₒ is the number of replicate determinations.

Within-sample standard deviation (swb) is derived by Formula (2):

$${s}_{within}^{2} = {M}_{within} \qquad ( 2 )$$

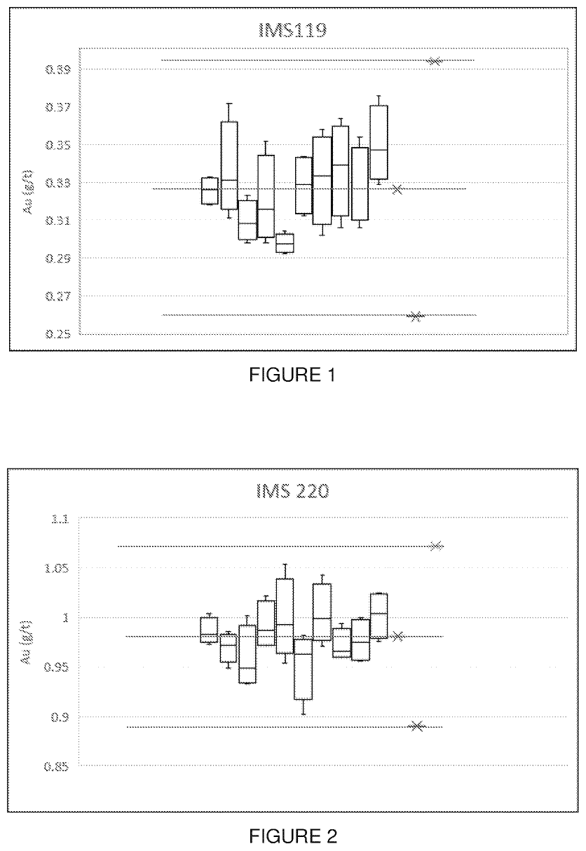

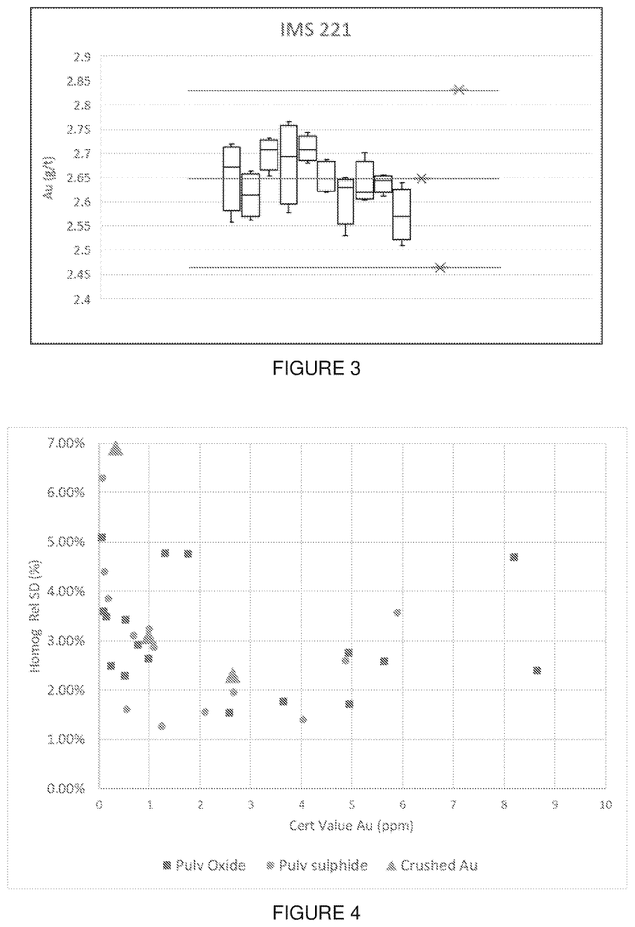

Ten (10)×500 g samples of each reference material were analysed by photon assay and the results are shown in FIGS. 1-3. The results are summarized in Table 3. The results indicate within-sample uncertainty (sᵣ, in this case equal to measurement uncertainty) is greater than the between-unit uncertainty (u_{bb}).

TABLE 3
Summary of between unit and within unit uncertainty
for 3 batches of crushed reference material.

| | IMS219 | IMS220 | IMS221 |
|---|---|---|---|
| Au (g/t) | 0.33 | 0.98 | 2.65 |
| u_{bb} | 0.011 | 0.012 | 0.035 |
| sᵣ | 0.019 | 0.027 | 0.050 |
| SD (total) | 0.023 | 0.030 | 0.061 |
| Rel SD % | 6.9% | 3.1% | 2.3% |

These results were compared against homogeneity study results conducted on traditional reference materials produced by Klen International. The results are shown in FIG. 4. The graph shows the distribution of homogeneity (relative standard deviation) as a function of the grade of gold (Cert Value ppm). A lower relative standard deviation for the same grade indicates a higher degree of homogeneity. The results show that the three products produced in Example 1 demonstrate similar results to commercially available reference materials having a much lower particle size.

### Example 2

The reference material of the present invention may be used to test the sub-sampling routine used in the sample preparation process prior to analysis. The sub-sampling routine should not bias with regard particle size, shape, or density. To test whether the reference material of the present invention could be used to identify sub-sampling error, particles of the reference material of the present invention where subjected to both the following sub-sampling routines:
i. 2 kg crushed reference material was sub-sampled into 4 lots of 500 g using a rotary sample divider (RSD) to establish a good sub-sampling routine; and
ii. 2 kg crushed reference material was sub-sampled into 4 lots of 500 g by pouring directly from a single bag to establish a poor sub-sampling routine.

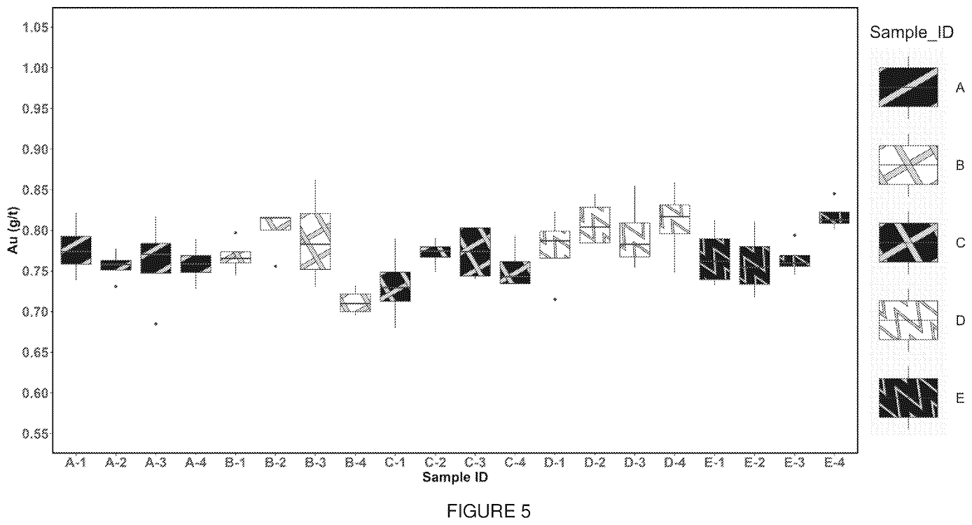

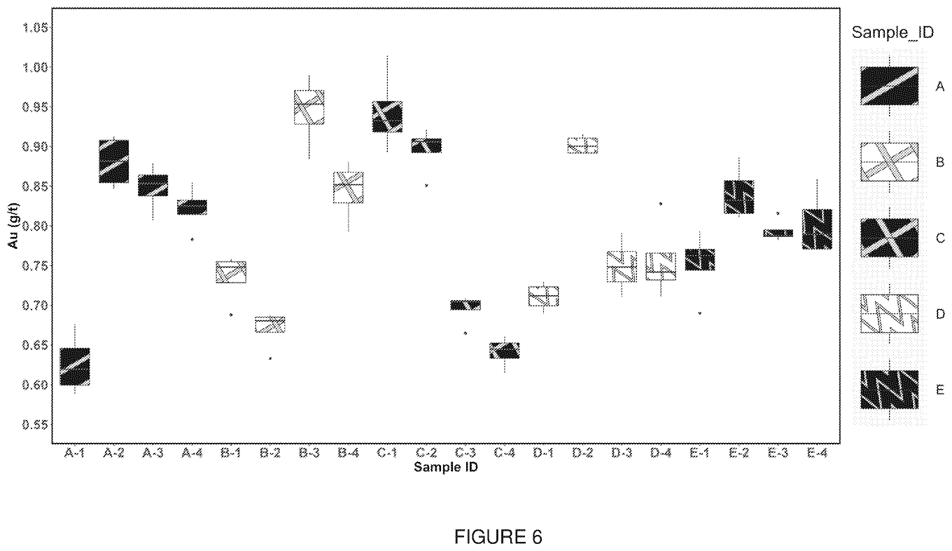

The resulting 500 g lots were repeat analysed by photon analysis and the results are displayed as box and whisker plots in FIG. 5 and FIG. 6, with the between-bottle (500 g unit) variance displayed in box plot pattern groups to denote their original 2 kg sample unit grouping. The between-bottle standard deviation (SD) in this case was 0.11 Au g/t for poured sub-sampled v 0.04 Au g/t for RSD sub-sampled when analysed using two-factor ANOVA with replication. This indicates that variance is about 3× higher with poor sub-sampling techniques over established robust methods. In this example about half the analysis determinations were over two standard deviations from the mean Au concentration for the poured sub-samples, compared to about 6% for the RSD sub-sampled units, as shown in Table 4. Values over two standard deviations from the mean are typically indicative of poor sub-sampling.

TABLE 4
Summary of sub-sampling technique performance

Sub-Sample Technique

| | Poured | RSD |
|---|---|---|
| SD between sub-sample | 0.112 | 0.039 |
| Overall SD | 0.099 | 0.040 |
| Count > 2*Overall SD_{RSD} | 41 | 5 |
| Suspect value % (>mean +/− 2SD) | 51% | 6% |

### Example 3

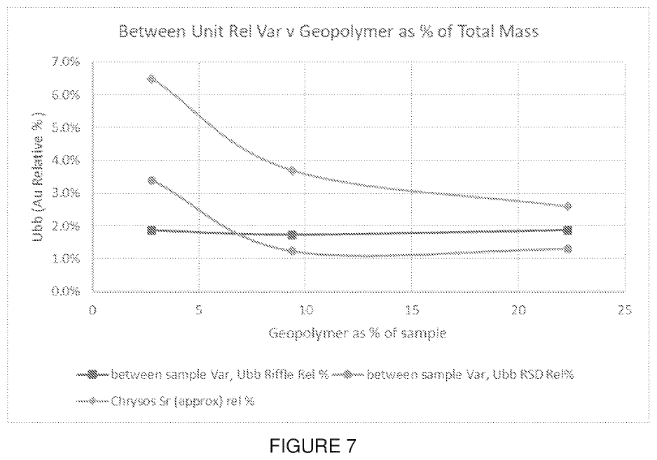

Trials were conducted to test whether the reference material of the present invention was suitable for use with riffle splitters. The reference materials produced in Example 1 were each subjected to a sub-sampling routine using a riffle splitter and compared to a RSD sub-sampling routine. The results are shown in FIG. 7. The results show that no significant difference between riffle splitter and RSD sub-sampling routines, indicating that riffle splitters are considered suitable for sub-sampling the reference material of the present invention.

### Example 4

A series of trials were conducted to test the effect of increasing dilution of the crushed reference material of the present invention with a filler material. Samples of the crushed material was mixed with barren basalt material at different dilution rates in PBS219 to PBS221. This resulted in increasing final Au concentration. Assessing the results required removing the effect of increasing relative measurement uncertainty from the between unit variance as displayed in FIG. 5, which demonstrate no significant change between 9% and 22% of geopolymer as total mass, and mixed results between ~2.8% and 9% of geopolymer as total mass.

### Example 5

Three separate batches of a reference material having different gold concentrations were prepared in accordance with the present invention. The respective batches targeted gold concentrations of 2.5 ppm (PBS232), 8 ppm (PBS233) and 22 ppm (PBS234). The gold was provided as an aqueous chloride solution (10,000 mg/L) purchased from Rowe Scientific. Each batch used fly-ash as the geopolymer binding reagent and a NaOH/Na-silicate is used as an activator solution. Each batch further comprises magnetite as an aggregate and colourant. The final composition of each batch is shown in Table 5.

TABLE 5
Batch Composition

| | PBS232 | PBS233 | PBS234 |
|---|---|---|---|
| Wet Weight (kg) | 415.7594 | 415.7594 | 395.4233 |
| Target Gold (g/T) | 2.5 | 8 | 24.8 |
| Fly Ash (kg) | 281.1325 | 281.1325 | 267.3815 |
| Magnetite (kg) | 15.83845 | 15.83845 | 15.06375 |
| 50% NaOH (kg) | 27.71729 | 27.71729 | 26.36156 |
| 42% Na-Sil (kg) | 71.27304 | 71.27304 | 67.78686 |
| 1% HCl (kg) | 19.79807 | 19.79807 | 18.82968 |
| Gold (g) | 0.864 | 2.7648 | 8.57088 |
| Au Volume (L) | 0.0864 | 0.27648 | 0.857088 |
| Au Sol Weight (kg) | 0.089338 | 0.28588 | 0.886229 |
| Au Sol Weight (g) | 89.3376 | 285.8803 | 886.229 |
| HCl 10% Excess (kg) | −1.89047 | −1.69393 | −0.99674 |
| Water Req (kg) | 19.70873 | 19.51219 | 17.94345 |
| Total Weight (kg) | 415.7594 | 415.7594 | 395.4233 |
| Gold ppm (g/T) | 2.078125 | 6.65 | 21.6752 |
| Dry Weight ( ) | 363.7895 | 363.7895 | 363.7895 |
| Gold ppm (g/T) | 2.375 | 7.6 | 23.56 |
| Target Dry Weight (kg) | 345.6 | 345.6 | 345.6 |
| Loss in Processing . . . (%) | 0.05 | 0.05 | 0.05 |
| Dry inc Losses (kg) | 363.7895 | 363.7895 | 363.7895 |
| Weight Reduction (%) | 0.125 | 0.125 | 0.08 |
| Wet Weight (kg) | 415.7594 | 415.7594 | 395.4233 |
| Target Gold (g/T) | 2.5 | 8 | 24.8 |

The fly-ash and magnetite added to a mixer and were stirred to ensure a homogenous dry mixture. Water was added to the dry mixture to dampen the powders and prevents dust generation. The activator solution was then added to the mixer to combine with the dry mixture. The mixer was operated until the slurry had a wet paste consistency and was free flowing. The gold solution was gradually added to the slurry and the mixer was operated for a further 15 minutes to ensure homogenous mixture.

The precursor slurry was then poured into tray moulds and placed into the oven at 100 °C for 24 hrs. After drying (curing) the contents of each tray were in a solid, hard rock type form. The material was removed from the trays, broken up with a hammer then loaded into 200 L drums. The material was crushed to −3 mm in a jaw crusher, and screened to −3 mm. The oversize (+3 mm) was recycled back through the crusher until all material passed 3 mm size.

The resulting solid material was then mixed with a ballast filler material of basalt, which had also been crushed and screened to −3 mm. The mixture targeted 380 kg of prepared material with 3076 kg of basalt to make a total batch size of 3456 kg. 12 empty drums were placed around a rotary splitter divider (RSD). The produced solid and the basalt filler were fed into the RSD in their required proportions. The material was distributed around each of the 12 drums equally, so each drum, or lot, contained 288 kg of final product with a similar blend of the two materials. The drums were then dismantled and the material feed back into the RSD 3 more times. The final product was segmented to prepare lots of 500 g and 2 kg. Each 500 g lot and 2 kg lot was placed into a labelled plastic bag. For each batch, 1728×500 g bags were packaged and 1296×2 kg bags were packaged. The samples prepared from each of the separate batches were numbered as follows:
IMS-235: PBS232+Basalt
IMS-236: PBS233+Basalt:
IMS-237: PBS234+Basalt

The resulting material was then tested for homogeneity using an external analysis laboratory. The laboratory was sent 15 samples of each of the 500 g packaged product and 2 kg packaged product and requested to analyse in duplicate. For the 2 kg samples, the laboratory prepared the sample by crushing, splitting and pulverising to a powder, followed by analysis for gold by fire assay (FA) method. For the 500 g samples, the laboratory loaded the entire sample in a jar and analysed for gold by the photon assay (PA) method. The result of the homogeneity study are shown in Table 6:

TABLE 6
Homogeneity Results

| | SAMPLE | | | | | |
|---|---|---|---|---|---|---|
| | IMS-235 | | IMS-236 | | IMS-237 | |
| Analytical Method | FA | PA | FA | PA | FA | PA |
| Number of Samples Submitted | 15 | 15 | 15 | 15 | 15 | 15 |
| Number of Samples tested | 15 | 15 | 15 | 15 | 15 | 15 |
| Total Samples in Analysis | 15 | 15 | 15 | 15 | 15 | 15 |
| No. Determinations per sample | 3 | 2 | 3 | 2 | 3 | 2 |
| Number of technically invalid | 0 | 0 | 0 | 0 | 0 | 0 |
| Mean concentration (Au g/t) | 0.24 | 0.23 | 0.74 | 0.74 | 2.14 | 2.1 |
| Material Standard Deviation (Au g/t) | 0.004 | 0.019 | 0.012 | 0.022 | 0.035 | 0.05 |
| Relative Standard Deviation | 1.72% | 8.12% | 1.57% | 3.05% | 1.65% | 2.37% |

The results demonstrated excellent homogeneity. Relative standard deviations for sample preparation and fire assay were all below 2% which is very low, and similar to the performance of pulverised reference materials of the prior art. The homogeneity results did not identify any nugget effects in the material. The higher standard deviations for photon assay are typical for the method for gold grades at these levels.

Characterisation of the samples was conducted at multiple external laboratories. A total of 10 laboratories received five 2 kg samples and were requested to conduct routine sample preparation, followed by fire assay for the determination of gold concentration. A total of 7 photon assay machines were used to analyse five 500 g samples. No additional preparation was required for photon assay, and samples were analysed in their entirety. The property values, and associated uncertainties from the characterisation studies are shown in Table 7.

TABLE 7
Crushed Gold CRM Characterisation Study Results

| | Analysis | | | | | |
|---|---|---|---|---|---|---|
| | Sample Preparation and Pb | | | Photon Assay | | |
| | Fire Assay (2 kg sample) | | | (500 g sample) | | |
| | CRM | | | | | |
| | Certified | Within Lab | Relative | Certified | Within Lab | Relative |
| | (g/t) | SD (g/t) | SD (%) | (g/t) | SD (g/t) | SD (%) |
| IMS-235 | 0.23 | 0.006 | 2.6 | 0.22 | 0.025 | 11.4 |
| IMS-236 | 0.72 | 0.024 | 3.3 | 0.72 | 0.028 | 3.9 |
| IMS-237 | 2.08 | 0.063 | 3.0 | 2.07 | 0.072 | 3.5 |

The characterisation results show good agreement between fire assay and photon assay for the multi-laboratory exercise. The results demonstrate the materials are fit for purpose as quality control tools, with relative standard deviations below 4%. The exception was the results of the low-level grade sample from photon assay where the variance of the measurement method dominates the uncertainty calculation as the grade approaches the detection limit. The relative standard deviation of 3-4% is higher than those obtained during the homogeneity study, and higher than typical pulverised CRMs (1-2%). This was attributed to the contribution to variance from the 10 laboratories who processed the samples, possibly due to differences in sample preparation and fire assay method.

### Example 6

An investigation was undertaken to understand the causes of variability between target concentrations measured by different analytical laboratories.

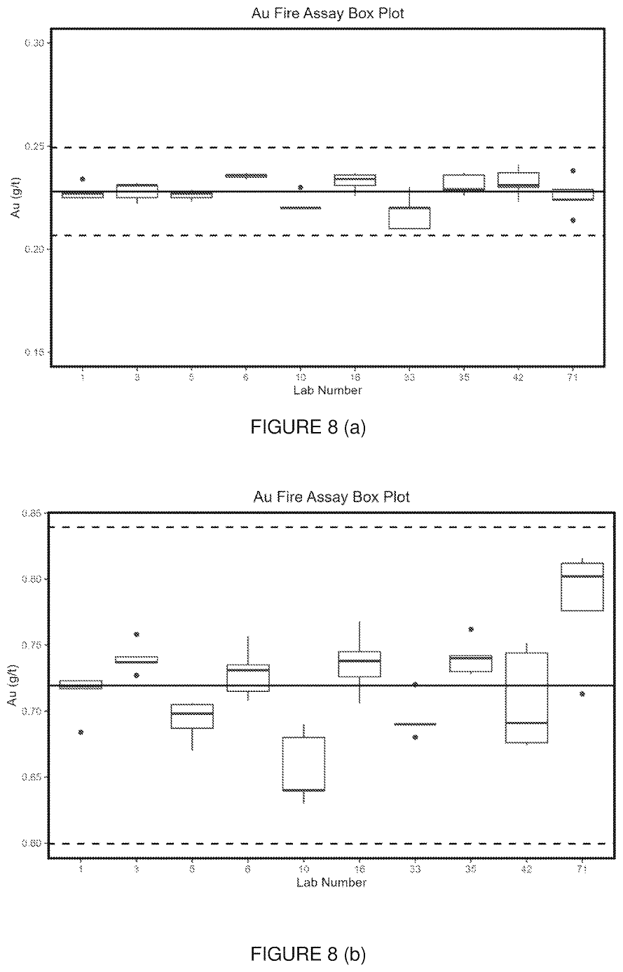

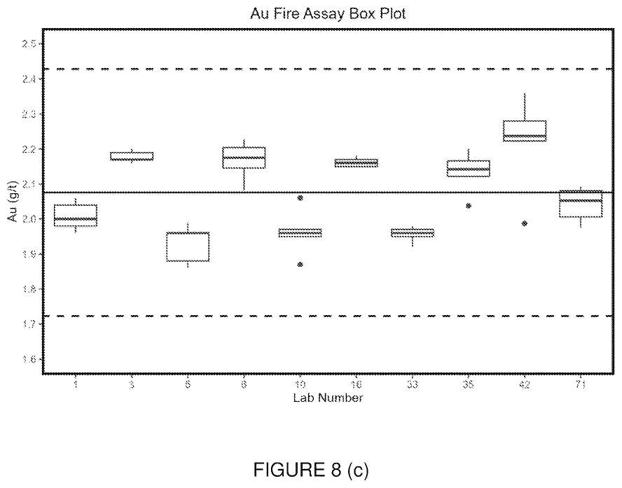

The reference material of the present invention is intended for monitoring and testing the accuracy and precision of sample preparation and sub-sampling procedures, followed by analysis. An investigation was undertaken to understand if the causes of variability between target concentrations measured by different analytical laboratories resulted from systematic biases in sample preparation and whether the reference material of the present invention could be used to identify them. Five samples of the material prepared in Example 5 were provided to 11 different laboratories for assay using fire assay. Laboratories prepared samples by splitting, if required, followed by pulverisation and subsampling. The results of these trials for each IMS-235, IMS-236 and IMS-237 are displayed in FIG. 8(a)-(c).

The results show some differences in the measured gold concentration across each of the labs. While not as evident in the lower grade, as the grade of the CRM increases, there appears to be two populations of data. The spread of the data explains why the standard deviations of the characterisation studies are significantly higher (2.6-3.3%) than the homogeneity studies (<2%). Some laboratories have reported results above the mean, and others below. The trend appears to be similar for all three grades, but more pronounced at the highest grade. In the case of IMS-237 (FIG. 8c), the z-scores for sample preparation fire assay method of the individual laboratory means were calculated using the global mean and standard deviation from the photon assay results. The results suggest for two of the laboratories, the bias is significant (z-score>2).

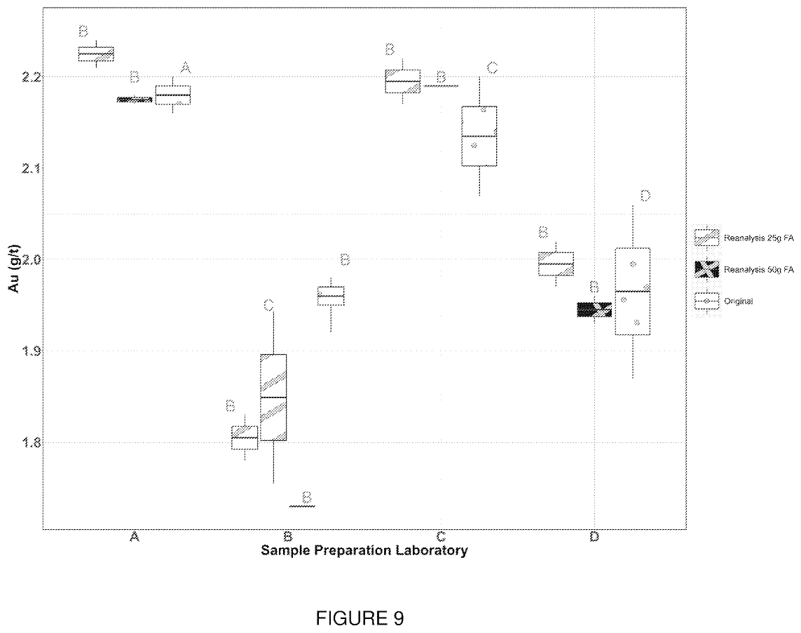

To test if the bias is occurring in sample preparation, or during analysis, pulverised samples were returned from 4 of the laboratories and subsequently labelled as A, B, C and D respectively. The selection was made from 4 laboratories where biases were both positive and negative. The returned pulverised samples prepared at laboratories A, B, C and D, were then sent to an alternate single laboratory for analysis. The results are shown in FIG. 9. The samples were re-numbered and submitted blindly and routinely to the alternate re-analysis laboratory without reference to the nature of the investigation.

The original results are shown in box plots with a dot pattern (the furthest box plot to the right of each laboratory indicated on the x-axis) and represents the original analysis for the submitted samples. In these cases, sample preparation and fire assay analyses were conducted at the same laboratory in the same submitted batch. The original results from laboratories A and C biased high compared to the mean, and laboratories B and D where biasing low. All of the pulverised samples returned were then submitted to laboratory B for repeat analysis. To complete the re-analysis exercise, pulverised samples prepared at laboratory B were also sent to laboratory C for analysis. Repeat analysis results for 25 g fire assay are shown in box plots with a stripe patten (first box from the left for each preparing laboratory), and results for 50 g fire assay are shown in box plots with the cross patten (second box from the left for each preparing laboratory).

The more significant trend in the box plots from FIG. 9 is that the repeat analysis at laboratory B and C matches the bias that occurred in the analysis from where the original samples were prepared. In addition, as a quality control check, the repeat analyses at laboratory B, for the resubmitted samples are consistent in bias with the original results prepared at laboratory B. This informs us that the analysis performed at laboratory B is providing consistent results, and there are biases occurring from analytical batch to batch. To determine if the overall high/low bias was occurring in sample preparation, or in the analysis, the alignment between the repeat analysis (stripe pattern and cross pattern box plots), and the identification of the preparing laboratory on the x-axis should be noted. If the bias was occurring in the analysis, one would expect the re-submitted samples to more closely align with the original results from where the samples were first prepared. However, the repeat analyses in the case of laboratory B and C more closely aligns with the origin of the prepared samples in both cases, therefore indicating a systematic bias has occurred in sample preparation.

The reference material of the present invention is provided in a large particle size with a high degree of homogeneity. This allows the reference material to be subjected to the same sample preparation and sub-sampling procedures that the sample undergoes. It is envisaged that the incorporation of the reference material of the present invention into the sample preparation and sub-sampling procedures will allow any errors or biases in these procedures to be identified, thereby improving the overall accuracy of the measured concentration. Standard reference materials of the prior art are only provided at small particle sizes. This means that they are unsuitable for use in the sample preparation and sub-sampling processes, meaning that they do not allow for errors of biases in these procedures to be tracked. Furthermore, the reference material of the present invention allows for the majority of targets to be homogenously distributed throughout the final material at any desired concentration. This is unable to be replicated using large particles of natural ores, especially for target materials that are nuggety, such as gold.

1. A method for the production of a reference material, the method comprising the steps of:
combining at least one target substance, one or more binding reagents and an activator solution to form a precursor slurry;
curing the precursor slurry to form a solid material; and
crushing the solid material to produce a reference material.

2. A method according to claim 1, wherein the target substance contains a target metal.

3. A method according to claim 1, wherein the one of more target substances are provided as a solid material.

4. A method according to claim 1, wherein the one or more target substances are provided in a carrier solution.

5. A method according to claim 4, wherein the target substances are solubilised in the carrier solution.

6. A method according to claim 1, wherein the target substance is a constituent of at least one of the binding reagents.

7. A method according to claim 6, wherein the binding reagent is a pulverised natural ore comprising the target substance.

8. A method according to claim 6, wherein the binding reagent is a pulverised synthetic material comprising the target substance.

9. A method according to claim 1, wherein the binding reagents are selected from one or more of geopolymer composite binders, hydraulic binders and refractory cement binders.

10. A method according to claim 1, wherein the activator solution is an aqueous solution.

11. A method according to claim 1, wherein one or more aggregates are included in the precursor slurry.

12. A method according to claim 11, wherein the target substance is a constituent of at least one of the aggregates.

13. A method according to claim 12, wherein the aggregate is a pulverized natural ore comprising the target substance.

14. A method according to claim 12, wherein the aggregate is a pulverised synthetic material comprising the target substance.

15. A method according to claim 1, wherein the precursor slurry is subjected to a mixing step.

16. A method according to claim 1, wherein curing the precursor slurry comprises a drying step.

17. A method according to claim 16, wherein the drying step is conducted at elevated temperatures.

18. A method according to claim 1, wherein curing the precursor slurry comprises an activation step.

19. A method according to claim 1, wherein the crushing step further comprises a sizing step.

20. A method according to claim 1, wherein the crushed material may be combined with a filler material.

21. (canceled)
